(19) United States
(12) Patent Application Publication
(10) Pub. No.: US 2026/0232944 A1
(43) Pub. Date: Aug. 13, 2026

(54) A VENTILATION SYSTEM WITH OXYGEN PREFILL COMPARTMENT

(71) Applicant: MAQUET CRITICAL CARE AB, Solna (SE)

(72) Inventor: Carl-Erik TROILI, Danderyd (SE)

(21) Appl. No.: 19/159,235

(22) PCT Filed: Feb. 16, 2024
(86) PCT No.: PCT/SE2024/050160

§ 371 Date: Aug. 22, 2025

(30) Foreign Application Priority Data
Feb. 23, 2023 (SE) 2350204-0

(51) Int. Cl. A61M 16/12; A61M 16/00; A61M 16/10; A61M 16/20

(52) U.S. Cl. CPC A61M 16/125; A61M 16/0066; A61M 16/022; A61M 16/1005; A61M 16/107; A61M 16/204; A61M 2016/1025; A61M 2202/0208; A61M 2205/42

(57) ABSTRACT

The disclosure relates to a ventilation system for supplying oxygen-containing breathing gas to a patient via a gas delivery line comprising a flow generator for generating a flow of air from an air inlet of the gas delivery line towards the patient. The system comprises an oxygen prefill compartment arranged downstream of the air inlet such that air from the air inlet flows through the oxygen prefill compartment on its way towards the patient. The system is configured such that oxygen may be delivered both to the patient and to the oxygen prefill compartment via an oxygen valve. By delivering oxygen to the oxygen prefill compartment prior to start of a high-flow period of an inspiration phase, the system is able to deliver up to 100% oxygen to the patient even with an oxygen valve that is incapable of delivering a flow of oxygen that matches the peak inspiratory flow generated by the flow generator.

### CROSS REFERENCE TO RELATED APPLICATIONS

The present application is a U.S. National Phase of International Application No. PCT/SE2024/050160 entitled “A VENTILATION SYSTEM WITH OXYGEN PREFILL COMPARTMENT”, and filed on Feb. 16, 2024. International Application No. PCT/SE2024/050160 claims priority to Swedish Patent Application No. 2350204-0 filed on Feb. 23, 2023. The entire contents of each of the above-listed applications are hereby incorporated by reference for all purposes.

### TECHNICAL FIELD

The present disclosure relates to a ventilation system for supplying oxygen-containing breathing gas to a patient, and a method and a computer program for controlling an oxygen content of the oxygen-containing breathing gas, as set forth in the introductory parts of the independent claims appended hereinafter.

### BACKGROUND ART

A blower-type ventilation system is typically configured to ventilate a patient by supplying a breathing gas mixture comprising air and oxygen to the airways of the patient. The blower of the ventilation system typically generates a gas flow that causes air to be withdrawn from the surroundings of the ventilation system via an air inlet of a gas delivery line for conveying the breathing gas mixture to the patient. In the gas delivery line, the air may be mixed with oxygen from an oxygen source, supplied to the gas delivery line via a controllable oxygen valve. The air and oxygen is typically mixed in a mixing chamber of the ventilation system to obtain a homogenous breathing gas mixture, and the oxygen valve is typically controlled such that the oxygen concentration of the breathing gas mixture corresponds to a set target oxygen concentration. Examples of such blower-type ventilation systems are disclosed in US 2003/0084900 A1 and US 2016/0082220 A1.

The blower of a blower-type ventilation system is typically capable of generating peak flows of 180 lpm or more. Some blowers may even be capable of generating peak flows of 240 lpm or more. The high peak flow of the blower puts high demands on the oxygen valve since the oxygen valve must be capable of delivering a corresponding oxygen flow in order for the ventilation system to deliver 100% oxygen to the patient, which may sometimes be desired. Even for more moderate oxygen concentrations, the oxygen valve must be capable of delivering high flows of oxygen in order to maintain the set target oxygen concentration in the breathing gas reaching the patient, especially during high-flow periods of inspiration.

Oxygen valves capable of delivering such high flows are often bulky and expensive to manufacture. Especially for blower-type ventilation systems, which are often mobile and particularly intended for environments where a source of pressurized air is not commonly available, use of bulky and expensive system components should be avoided.

Consequently, there is a need for a ventilation system that allows use of a flow generator with high peak flows while being capable of delivering breathing gas with high oxygen concentrations without the use of high-capacity flow valves of other expensive system components.

### SUMMARY

It is an object of the present disclosure to mitigate, alleviate or eliminate one or more of the above-identified deficiencies and disadvantages in the prior art.

It is a particular object of the present disclosure to provide a ventilation system using a flow generator capable of generating and delivering high flows of oxygen-rich breathing gas to a patient without the need for high-capacity flow valves of other expensive system components.

These and other objectives that will become apparent in view of the detailed description following hereinafter are achieved by a ventilation system, a method and a computer program as defined in the appended claims.

According to one aspect of the present disclosure there is provided a ventilation system for supplying oxygen-containing breathing gas to a patient via a gas delivery line comprising a flow generator for generating a flow of air from an air inlet of the gas delivery line towards the patient. The ventilation system comprises an oxygen prefill compartment forming a part of the gas delivery line, arranged downstream of the air inlet such that air from the air inlet flows through the oxygen prefill compartment on its way towards the patient. The ventilation system further comprises a controllable oxygen valve for regulating a flow of oxygen through an oxygen inlet of the gas delivery line, which oxygen inlet is arranged in fluid communication with the oxygen prefill compartment, and a controller for controlling the oxygen valve such that a mean oxygen concentration of breathing gas delivered to the patient during inspiration substantially corresponds to a set target oxygen concentration. The controller is further configured to control the oxygen valve such that the oxygen prefill compartment is prefilled with a volume of oxygen prior to start of a high-flow period of an inspiration phase.

By prefilling the oxygen prefill compartment with a volume of oxygen prior to start of the high-flow period of the inspiration phase, high concentrations of oxygen (up to 100%) can be delivered to the patient even if the oxygen valve is unable to deliver a flow of oxygen that matches the inspiratory peak flow generated by the flow generator.

This is most easily understood by studying an exemplary scenario where the set target oxygen concentration is 100%. In this case, in a ventilation system without an oxygen prefill compartment, air would inevitably be drawn into the gas delivery line via the air inlet and delivered to the patient when the flow of oxygen from the oxygen valve falls below the inspiratory flow generated by the flow generator. Therefore, such a ventilation system would not be able to deliver 100% oxygen unless the oxygen valve is capable of delivering a flow of oxygen matching the inspiratory peak pressure of the flow generator. By using and prefilling the proposed oxygen prefill compartment with a volume of pure oxygen prior to the high-flow period of the inspiration phase, the flow required to compensate for the difference in flow between the flow generated by the flow generator and the maximum flow through the oxygen valve during the high-flow period will be withdrawn from the oxygen prefill compartment, thereby enabling a breathing gas consisting of 100% oxygen to be delivered to the patient.

Consequently, the proposed ventilation system enables use of an oxygen valve that does not match the capacity of the flow generator in terms of maximum flowrate. Thereby, a less complex, bulky and expensive oxygen valve can be used with a maintained peak inspiratory flow capacity of the ventilation system.

The flow generator and the oxygen valve may hence advantageously be configured such that the gas flow generated by the flow generator during the high-flow period of the inspiration phase exceeds a maximum flow of oxygen that can be delivered via the oxygen valve. In some embodiments, the flow generator may be a high-flow generator capable of generating a gas flowrate of at least 120 lpm, preferably at least 180 lpm and most preferably at least 240 lpm, whereas the oxygen valve may be capable of delivering a maximum oxygen flow of no more 60 lpm.

In a situation where the oxygen concentration of breathing gas delivered to the patient falls below the set target oxygen concentration, the controller may be configured to first apply an oxygen valve control strategy according to which the oxygen concentration of the breathing gas is increased above the set target oxygen concentration during at least one low-flow period of one or more inspiration phases to come. A low-flow period, as used herein, is a period during which the gas flow generated by the flow generator is less than the maximum flow of oxygen deliverable via the oxygen valve. If increasing the oxygen concentration above the set target oxygen concentration during low-flow periods is not enough to reach the set target oxygen concentration, or if the oxygen concentration of the breathing gas cannot be further increased during low-flow periods (e.g. when delivering 100% oxygen), the controller switches to an oxygen valve control strategy according to which the oxygen valve is controlled such that the oxygen prefill compartment is prefilled with oxygen prior to start of a high-flow period of one or more inspiration phases to come. In other words, the controller may be configured to control the oxygen valve such that the oxygen prefill compartment is prefilled with oxygen prior to start of a high-flow period of at least one inspiration phase in response to a failure of making the mean oxygen concentration substantially correspond to the set target oxygen concentration by controlling the oxygen valve such that the oxygen concentration of the breathing gas is increased above the set target oxygen concentration during at least one low-flow period of at least one inspiration phase.

The controller may be configured to control the oxygen valve such that the oxygen prefill compartment is prefilled with a volume of oxygen during a low-flow period of a breathing cycle. The low-flow period of the breathing cycle may occur during any of, or any combination of, a final phase of an inspiration phase, an initial phase of an inspiration phase, and an expiration phase. Consequently, in situations where the oxygen prefill compartment must be used to reach the set target oxygen concentration during an inspiration phase, the controller may control the oxygen valve such that the oxygen prefill compartment is prefilled during a final phase of a preceding inspiration phase, during the expiration phase preceding the inspiration phase, during an initial phase of the inspiration phase itself, or any combination thereof. The oxygen inlet of the gas delivery line may be arranged in relation to the oxygen prefill compartment such that an excess flow of oxygen exceeding a current gas flow generated by the flow generator flows into the oxygen prefill compartment. This way, the oxygen prefill compartment can be prefilled with oxygen during any period for which the inspiratory flow generated by the flow generator is less than the maximum flow of oxygen deliverable via the oxygen valve.

The controller may be configured to determine whether to prefill the oxygen prefill compartment based on a measured oxygen concentration of breathing gas delivered to the patient and the set target oxygen concentration. The controller may, for example, be configured to determine the mean oxygen concentration of breathing gas delivered to the patient during one or more previous breaths based on the measured oxygen concentration, and determine whether to prefill the oxygen prefill compartment prior to start of the high-flow period of one or more coming inspiration phases based on a comparison between the mean oxygen concentration and the set target oxygen concentration. If, for example, the mean oxygen concentration delivered during one or more previous breaths falls below a threshold value (which may correspond to the set target oxygen concentration) or is gradually decreasing over the course of a plurality of previous breaths, the controller may determine to increase the mean oxygen concentration of the breathing gas to be delivered during coming inspiration phases by controlling the oxygen valve such that the oxygen prefill compartment is prefilled with a volume of oxygen prior to start of the high-flow period of the one or more coming inspiration phases.

According to some embodiments, the oxygen prefill compartment comprises an elongated gas passageway serving as an oxygen reflector and configured such that oxygen that flows into the oxygen prefill compartment from the oxygen inlet pushes air contained in the gas passageway out from the oxygen prefill compartment in the direction of the air inlet, and such that air that flows into the oxygen prefill compartment from the air inlet pushes oxygen contained in the gas passageway out from the oxygen prefill compartment in the direction of the patient. This way, oxygen may be delivered to the oxygen prefill compartment, maintained in the oxygen prefill compartment, and delivered to the patient from the oxygen prefill compartment without the need for upstream or downstream valve arrangements, pressurized gas tanks, or other expensive or energy-consuming circuit components. The oxygen prefill compartment may hence form a non-pressurized, open-ended gas passageway serving as an oxygen reflector for receiving and maintaining a volume of oxygen during low-flow periods of ventilation, and for reflecting the volume of oxygen back towards the patient during high-flow periods of ventilation.

According to some embodiments, the oxygen inlet is arranged downstream of the elongated gas passageway, or in a downstream end of the elongated gas passageway. This way, a well-defined gas front is formed between the oxygen and the air in the oxygen prefill compartment and mixing of oxygen and air is prevented or at least mitigated, thereby preventing oxygen from leaking out of the air inlet and thus improving oxygen delivery control. Furthermore, the well-defined gas front between oxygen and air in the oxygen prefill compartment serves to effectively push the oxygen and air back and forth in the oxygen prefill compartment while minimizing mixing of the gases. Yet further, the downstream location of the oxygen inlet makes oxygen flow into the oxygen prefill compartment only when the flow through the oxygen valve exceeds the current flow generated by the flow generator. This way, the controller can control the degree of prefilling of the oxygen prefill compartment by controlling the flow of oxygen through the oxygen valve in relation to the current inspiratory flow generated by the flow generator.

According to some embodiments, the elongated gas passageway of the oxygen prefill compartment has a length that is at least five times a width or a diameter of the elongated gas passageway. Preferably, the elongated gas passageway has a length that is at least ten times its width or diameter. This way, a laminar flow is obtained in the oxygen prefill compartment, which further facilitates preservation of the well-defined gas front between the oxygen and air gas pillars moving back and forth in the oxygen prefill compartment, and so further prevents mixing of oxygen and air.

The dimensions of the elongated gas passageway may be adapted to the requirements of the ventilated patient. Typically, the elongated gas passageway has a volume of 200-3000 ml. According to some embodiments, the elongated gas passageway of the oxygen prefill compartment has a volume of 200-2000 ml, preferably 250-1500 ml, more preferably 300-1000 ml, and most preferably 400-800 ml.

The controller may be configured to prefill whole or parts of the oxygen prefill compartment with oxygen prior to start of the high-flow period of the inspiration phase. The volume of oxygen with which the oxygen prefill compartment is prefilled prior to start of the high-flow period of inspiration (hereinafter referred to as the prefill oxygen volume) should correspond at least to the volume of oxygen that needs to be delivered to the patient during the high-flow period in order for the mean oxygen concentration of the breathing gas delivered during the entire inspiration phase to reach the set target oxygen concentration. Consequently, the controller may be configured to determine the prefill oxygen volume based on the flow profile of the inspiratory flow generated by the flow generator and the set target oxygen concentration. Preferably, in order to deliver 100% oxygen to the patient, the prefill oxygen volume should correspond to, or exceed, a top-flow volume corresponding to the volume of breathing gas delivered to the patient at a flowrate exceeding the maximum flowrate of oxygen deliverable via the oxygen valve.

In some embodiments, the oxygen prefill compartment may consist of a length of tubing constituting the elongated gas passageway. In other embodiments, the oxygen prefill compartment may comprise a casing with an air inlet port for receiving air from the air inlet and an oxygen inlet port for receiving oxygen from the oxygen inlet. The air inlet port and the oxygen inlet port are fluidly coupled to each other via a length of tubing or a gas duct extending between the air inlet port and the oxygen inlet port, within the casing. The oxygen prefill compartment may be detachably connected in line with the gas delivery line between the air inlet and the oxygen inlet.

According to some embodiments, the oxygen prefill compartment and the oxygen inlet are arranged upstream of the flow generator along the gas delivery line. This is advantageous from a noise perspective. A flow generator, such as a blower, often generates a substantial amount of noise. Downstream of the flow generator, this noise is trapped within the gas delivery line and the tubing and the circuit components between the flow generator and the patient serve as a sound trap effectively preventing the noise from reaching high sound levels outside the gas delivery line. On the upstream-side of the flow generator, on the other hand, the noise escapes the gas delivery line via the air inlet, normally causing high sound levels in the area behind the flow generator. By placing the oxygen prefill compartment upstream of the flow generator, between the flow generator and the air inlet, the volume and the gas passageway of the oxygen prefill compartment will, in a similar manner as the tubing and circuit components downstream of the flow generator, serve as a sound trap having a damping effect on the noise.

According to another aspect of the present disclosure there is provided a method for controlling an oxygen content of oxygen-containing breathing gas delivered to a patient by a ventilation system comprising a flow generator for generating a flow of air from an air inlet of a gas delivery line towards the patient, via an oxygen prefill compartment of the gas delivery line, and a controllable oxygen valve for regulating a flow of oxygen through an oxygen inlet of the gas delivery line, which oxygen inlet is arranged in fluid communication with the oxygen prefill compartment. The method comprises the step of prefilling the oxygen prefill compartment with a volume of oxygen that is to be delivered to the patient during a subsequent high-flow period of an inspiration phase.

All features and advantages with regard to the ventilation system as disclosed herein is also applicable to the method for controlling an oxygen content of breathing gas delivered to a patient by such a ventilation system.

As clear from above, the method aims at controlling the oxygen-content of the breathing gas such that a mean oxygen concentration of breathing gas delivered to the patient during inspiration substantially corresponds to a set target oxygen concentration. As also clear from above, the step of prefilling the oxygen prefill compartment may be performed in response to a failure of making the mean oxygen concentration correspond to the set target oxygen concentration during one or more inspiration phases.

According to some embodiments, the oxygen inlet is arranged downstream of an elongated gas passageway of the oxygen prefill compartment, or in a downstream end of the elongated gas passageway, whereby the oxygen prefill compartment may be prefilled during a low-flow period during which the gas flow generated by the flow generator is lower than the maximum flow of oxygen that can be delivered via the oxygen valve.

According to some embodiments, the low-flow period occurs during any of, or any combination of, a final phase of an inspiration phase, an initial phase of an inspiration phase, and an expiration phase.

According to some embodiments, the step of prefilling the oxygen prefill compartment prior to start of the high-flow period of the at least one inspiration phase is performed in response to a failure of making the mean oxygen concentration substantially correspond to the set target oxygen concentration by increasing the oxygen concentration of the breathing gas above the set target oxygen concentration during at least one low-flow period of at least one inspiration phase.

The method may be a computer-implemented method performed by the above-mentioned controller for controlling the oxygen valve of the ventilation system upon execution of a computer program.

Thus, according to another aspect of the present disclosure, there is provided a computer program for controlling an oxygen content of breathing gas delivered to a patient by a ventilation system comprising a flow generator for generating a flow of air from an air inlet of a gas delivery line towards the patient, an oxygen prefill compartment forming part of the gas delivery line, arranged downstream the air inlet such that air from the air inlet flows through the oxygen prefill compartment on its way towards the patient, a controllable oxygen valve for regulating a flow of oxygen through an oxygen inlet of the gas delivery line, which oxygen inlet is arranged in fluid communication with the oxygen prefill compartment, and a controller for controlling the oxygen valve such that a mean oxygen concentration of breathing gas delivered to the patient during an inspiration phase substantially correspond to a set target oxygen concentration. The computer program comprises computer-readable instructions which, when executed by a processor of the controller, causes the controller to perform the above described method.

According to yet another aspect of the present disclosure, there is provided a computer program product comprising a non-transitory data storage medium storing the computer program.

The present disclosure will become apparent from the detailed description following hereinafter. The detailed description and specific examples disclose preferred embodiments of the disclosure by way of illustration only. Those skilled in the art understand from guidance in the detailed description that changes and modifications may be made to the teachings of the disclosure within the scope of the appended claims.

### BRIEF DESCRIPTIONS OF THE DRAWINGS

The above objects, as well as additional objects, features and advantages of the present disclosure, will be more fully appreciated by reference to the following illustrative and non-limiting detailed description of example embodiments of the present disclosure, when taken in conjunction with the accompanying drawings.

FIG. 1 illustrates a ventilation system for supplying oxygen-containing breathing gas to a patient, according to an exemplary embodiment of the present disclosure.

FIG. 2 illustrates a ventilation system for supplying oxygen-containing breathing gas to a patient, according to another exemplary embodiment of the present disclosure.

FIG. 3 illustrates a ventilation system for supplying oxygen-containing breathing gas to a patient, according to yet another exemplary embodiment of the present disclosure.

FIG. 4 illustrates an oxygen prefill compartment component of the ventilation system, according to an exemplary embodiment of the present disclosure.

FIG. 5 is a flowchart illustrating a method for controlling an oxygen content of oxygen-containing breathing gas delivered to a patient, according to an exemplary embodiment of the present disclosure.

### DETAILED DESCRIPTION

The present disclosure will now be described with reference to the accompanying drawings, in which preferred example embodiments of the disclosure are shown. The disclosure may, however, be embodied in other forms and should not be construed as limited to the herein disclosed embodiments. The disclosed embodiments are provided merely to fully convey the scope of the disclosure to the skilled person.

It is to be understood that the terminology used herein is for purpose of describing particular embodiments only, and is not intended to be limiting. It should be noted that, as used in the specification and the appended claims, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the terms “comprising”, “including”, “containing” and similar wordings are intended to be open-ended transitional terms that do preclude the possibility of additional elements or steps.

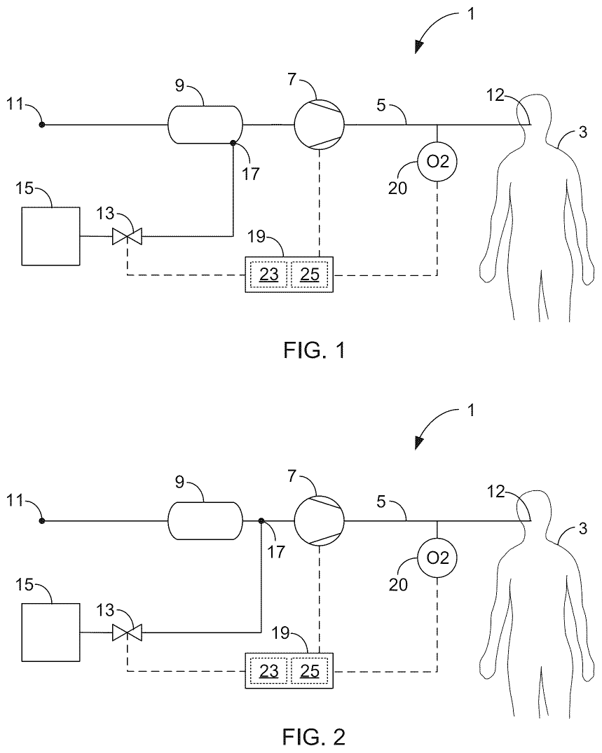

FIG. 1 schematically illustrates a ventilation system 1 for supplying oxygen-containing breathing gas to a patient 3. In particular, the ventilation system 1 is configured for delivery of a breathing gas comprising air, oxygen or a mixture of air and oxygen. The ventilation system 1 comprises a gas delivery line 5 comprising a flow generator 7 for generating a flow of air from an air inlet 11 of the gas delivery line 5 towards the patient 3. The breathing gas is delivered to the airways of the patient 3 via a patient connector 12, which may comprise a face mask, a tracheal tube, a nasal cannula, or any other type of patient connector known in the art of mechanical ventilation. The gas delivery line 5 is an inspiratory limb of the ventilation system 1 for conveying the breathing gas, also herein referred to as inspiratory gas, to the patient 3. The ventilation system 1 typically also comprises an expiratory limb including ventilation circuit components such as an expiratory valve, one or more flow and pressure sensors, etc., which expiratory limb has been omitted from the drawings so as not to obscure them with unnecessary details.

The air inlet 11 constitutes an open end of the gas delivery line 5 arranged in direct communication with air surrounding the ventilation system 1, such that surrounding air is drawn into the gas delivery line 5 via the air inlet 11 upon operation of the flow generator 7 creating an under pressure in the gas delivery line 5. An air filter (not shown) is typically positioned downstream of the air inlet 11 in the gas delivery line 5 for filtering the air entering the ventilation system 1. The air inlet 11 constitutes an upstream-end of the gas delivery line 5. The directional terms “upstream” and “downstream” herein refers to a gas flow through the gas delivery line 5 flowing in the direction of the patient 3, meaning that the air inlet 11 constitutes an upstream-end of the gas delivery line 5, whereas the patient connector 12 constitutes a downstream-end of the gas delivery line 5.

The flow generator 7 may be any type of flow generator that is able to generate an adequate inspiratory flow or air from the air inlet 11 towards the patient 3, without the need for any additional flow-generating components or sources for pressurised air. In the illustrated example, the flow generator 11 is a blower. The term “blower” is herein intended to encompass the terms “fan”, “compressor” and “turbine” which are exemplary terms for flow-generating devices often interchangeably used with “blower” in the field of mechanical ventilation. Likewise, the term “blower” is intended to encompass various types of blower arrangements, including, but not limited to, conventional ventilator blower arrangements and piezoelectric micro-blowers. The ventilation system 1 may hence constitute what is sometimes referred to as a blower-type ventilation system or blower based ventilation system, a blower-type ventilation or blower based ventilator, or simply a blower ventilator. The flow generator 7 is preferably configured to generate a peak inspiratory flowrate of at least 120 litres per minute (lpm), more preferably at least 180 lpm, and most preferably at least 240 lpm.

The ventilation system 1 further comprises a gas reservoir, herein functionally referred to as an oxygen prefill compartment 9, constituting a part of the gas delivery line 5. The oxygen prefill compartment 9 is arranged downstream of the air inlet 11 such that air that is drawn into the air inlet 11 from the surroundings of the ventilation system 1 by the operation of the flow generator 7 flows through the oxygen prefill compartment 9 on its way towards the patient 3. In the illustrated embodiment, the oxygen prefill compartment 9 is arranged upstream of the flow generator 7. In other embodiments, the oxygen prefill compartment 9 may be arranged downstream of the flow generator 7.

The ventilation system 1 further comprises a controllable oxygen valve 13 for regulating a flow of oxygen through an oxygen inlet 17 of the gas delivery line 5. The oxygen inlet 17 is arranged in fluid communication with the oxygen prefill compartment 9 and is preferably located downstream of the oxygen prefill compartment 9 or in a downstream-end of the oxygen prefill compartment, as will be discussed in more detail below. The oxygen is supplied from an oxygen source 15, which may be an oxygen outlet of a hospital facility, a pressurised bottle of oxygen, or any other type of oxygen source suitable of delivering oxygen for medical applications. The oxygen valve 13 is typically a low-flow valve which, in this context, means that the flow capacity of the oxygen valve 13 is less than the flow capacity of the flow generator 7. The oxygen valve 13 is preferably configured such that it is able to deliver a maximum flow of no more than 60 lpm at valve inlet pressures between 4-5 bar. This flowrate allows the oxygen valve to deliver a constant oxygen concentration during the entire inspiration phase for all breaths defined in Table 201.104 of ISO 80601-2-12:2020, while ensuring that the flowrate will never exceed 60 lpm averaged over 10 seconds at a pressure of 280 kPa, measured at the oxygen inlet 17 of the gas delivery line 5, as required by the 201.4.11.101.2 Compatibility requirement for ventilators conforming with ISO 7396-1:2016. Besides allowing use of a small, cost-efficient and energy-efficient oxygen valve that fulfils the Compatibility requirement by virtue of its technical limitations, the maximum flowrate of 60 lpm improves patient safety since it eliminates the risk of delivering really high flowrates to the patient in the unexpected and potentially harmful event of valve malfunctioning.

Notably, the ventilation system 1 does not comprise any valves or pressure-regulating arrangements for controlling the pressure in the oxygen prefill compartment 9. The oxygen prefill compartment 9 is always in fluid communication with surrounding air via the air inlet 11 and gas is allowed to flow freely through the oxygen prefill compartment 9 during both inspiration and expiration. This way, an open, non-pressurized ventilation system 1 with a minimum of complex, bulky and energy-consuming system components is obtained.

The ventilation system 1 further comprises a control module or controller 19 for controlling the oxygen valve 13. By controlling the oxygen valve 13, the controller 19 indirectly controls the flowrate of oxygen flowing through the oxygen valve 13. The controller 19 is typically a computerised controller that controls the oxygen valve 13 by following computer-readable instructions. Unless stated otherwise, any step involving control of the oxygen valve 13 described hereinafter is performed by the controller 19 upon execution of a computer program comprising the computer-readable instructions by a processor 23 of the controller. The computer program may be stored in a data storage medium 25 of the controller 19, such as a non-transitory memory hardware device.

The controller is configured to control the oxygen valve 13 such that the oxygen concentration of breathing gas delivered to the patient 3 during inspiration substantially corresponds to a set target oxygen concentration. As will be described in more detail below, this does not necessarily mean that the momentary oxygen concentration is constant and corresponds to the set target oxygen concentration at any given time during the inspiration phase. Instead, it means that at least a mean oxygen concentration of the breathing gas delivered during the whole inspiration phase, or a mean oxygen concentration of breathing gas delivered during a plurality of inspiration phases, substantially corresponds to the set target oxygen concentration, i.e. that the volumetric concentration of oxygen in the breathing gas delivered to the patient during the course of one or more inspiration phases substantially corresponds to the set target oxygen concentration.

In accordance with the principles of the present disclosure, in order for the ventilation system 1 to deliver large tidal volumes with high oxygen concentration at with high peak inspiratory flowrates, the controller is configured to selectively make use of the oxygen prefill compartment 9 by controlling the oxygen valve 13 such that the oxygen prefill compartment 9 is prefilled with a volume of oxygen prior to start of a high-flow period of an inspiration phase. A high-flow period, as used herein, is a period during which the gas flow generated by the flow generator 7 is higher than the maximum flow of oxygen deliverable via the oxygen valve 13.

The controller 19 may be configured to detect an undesired decrease in delivered oxygen concentration, e.g. by detecting if the oxygen concentration delivered to the patient 3 falls below a predetermined threshold value, which may correspond to the preset target oxygen concentration or a value below a predetermined tolerance range of the target oxygen concentration, or if the delivered oxygen concentration decreases over time. The undesired decrease in delivered oxygen concentration may be detected based on oxygen measurements obtained by an oxygen sensor 20. The oxygen sensor 20 is typically arranged downstream of the flow generator 7 and may, for example, be arranged in or close to a Y-piece connecting the illustrated inspiratory limb of the ventilation system 1 with an expiratory limb (not shown). When the controller 19 detects an undesired decrease in delivered oxygen concentration, it may be configured to prefill the oxygen prefill compartment 9 with oxygen prior to the high-flow period of one or more inspiration phases to come. This way, the controller 19 serves to prime the oxygen prefill compartment such that the ventilation system 1 is able to increase the oxygen concentration of breathing gas delivered during a subsequent high-flow period of inspiration.

The oxygen prefill compartment 9 may be prefilled with oxygen during a low-flow period during which the gas flow generated by the flow generator 7 is less than the maximum flow of oxygen deliverable via the oxygen inlet 17 by the oxygen valve 13. The low-flow period may occur during a final phase of an inspiration phase, an initial phase of an inspiration phase, an expiration phase, or any combination thereof. During the low-flow period, the controller 19 controls the oxygen valve 13 such that the flowrate of oxygen flowing through the oxygen valve 13 into the gas delivery line 5 via the oxygen inlet 17 exceeds the relatively low flowrate generated by the flow generator 7 during the low-flow period. A first part of the oxygen flow corresponding to the current gas flow generated by the flow generator 7 will then flow downstream in the gas delivery line 5 and further onto the patient 3, whereas a second part of the oxygen flow, constituting what may herein be referred to as an excess oxygen flow, will flow upstream in the gas delivery line 5, into the oxygen prefill compartment 9. The excess oxygen flow will push gas already contained in the oxygen prefill compartment 9 out of the compartment in the direction of the air inlet 11. By controlling the flow of oxygen through the oxygen valve 13 based on the current flowrate generated by the flow generator 7, the controller 19 may control the excess flow of oxygen flowing into the oxygen prefill compartment 9 and hence the prefill volume of oxygen with which the compartment is prefilled.

When, during a high-flow period following the priming of the oxygen prefill compartment 9, the inspiratory flowrate generated by the flow generator 9 exceeds the flowrate of oxygen delivered via the oxygen valve 13 (typically corresponding to a maximum oxygen flow deliverable by the oxygen valve 13), the prefill volume of oxygen will be withdrawn from the oxygen prefill compartment to make up for the difference between the flowrate generated by the flow generator 7 and the flowrate of oxygen delivered via the oxygen valve 13. This way, the mean oxygen concentration of inspiratory gas delivered to the patient 3 during the high-flow period can be controlled by prefilling the oxygen prefill compartment 9 with an oxygen prefill volume that is adapted to the volume of inspiration gas delivered to the patient during the high-flow period and the volume of oxygen delivered via the oxygen valve 13 during the high-flow period. As readily understood in view of the foregoing description, the ventilation system 1 may this way be able to deliver up to 100% oxygen to the patient 3 by, for each inspiration phase, prefilling the oxygen prefill compartment 9 with an oxygen prefill volume at least corresponding to the top-flow volume of inspiration gas that is delivered to the patient at flowrates exceeding the maximum flowrate of the oxygen valve 13.

As mentioned above, this “priming strategy” according to which the oxygen prefill compartment 9 is prefilled with oxygen before a subsequent high-flow period of an inspiration phase may be applied by the controller 19 in response to a failure of the mean oxygen concentration of delivered breathing gas to reach the set target oxygen concentration. The controller 19 may be configured to apply different oxygen valve control strategies depending on the set target oxygen concentration and/or the ability of the oxygen valve 13 to deliver the set target oxygen concentration given the flow profile of the inspiratory flow generated by the flow generator 7.

A first control strategy may be a conventional proportional control strategy according to which the oxygen valve 13 is controlled such that the flow of oxygen through the oxygen valve 13 is proportional to the current inspiratory flow generated by the flow generator 7. This way, a constant oxygen concentration substantially corresponding to the set target oxygen concentration will be delivered to the patient 3 throughout the entire inspiration phase as long as the oxygen valve 13 is able to deliver the proportional flow required to reach the set target oxygen concentration. If the controller 19 detects that the mean oxygen concentration of delivered breathing gas falls below the set target oxygen concentration, which is most likely due to the inability of the oxygen valve 13 to deliver a proportionate flow of oxygen matching the inspiratory flow during the high-flow period of inspiration, the controller 19 may switch to a second control strategy.

According to the second control strategy, the controller 19 controls the oxygen valve 13 such that the oxygen concentration delivered to the patient 3 during one or more low-flow periods of inspiration is higher than the set target oxygen concentration to compensate for the lower concentration of oxygen delivered during the high-flow period of inspiration. This way, the mean oxygen concentration of breathing gas delivered to the patient 3 during the inspiration phase as a whole, corresponding to the volumetric concentration of oxygen delivered to the patient as seen over the entire inspiration phase, can still be made to correspond to the set target oxygen concentration. For example, in accordance with this second control strategy, the controller 19 may be configured to control the oxygen valve 13 such that the oxygen concentration of the breathing gas is higher that the set target oxygen concentration during an initial and/or final phase of inspiration. If the controller detects that increasing the oxygen concentration above the set target oxygen concentration during low-flow periods of inspiration cannot be done (as will be the case if the set target oxygen concentration is 100%), or is not enough to reach a mean oxygen concentration substantially corresponding to the set target oxygen concentration, the controller 19 may switch to a third control strategy. This third control strategy may be the “priming strategy” according to which the oxygen valve 13 is controlled such that the oxygen prefill compartment 9 is prefilled with oxygen prior to start of the high-flow period of one or more inspiration phases, in accordance with the teachings of the present disclosure.

Preferably, the oxygen valve 13 is a control valve capable of delivering a variable flow of oxygen to the gas delivery line 5 via the oxygen inlet 17. In some embodiments, however, the oxygen valve 13 may be an on-off valve that either allows for unimpeded flow through the valve or acts to prevent flow altogether. In this scenario, the controller 19 can control the opening and closing times of the oxygen valve 13 such that flow pulses of oxygen is delivered to the gas delivery line 5 via the air inlet 17. The pulsed oxygen flow results in a mean oxygen flow through the oxygen valve 13, which mean oxygen flow may be controlled in accordance with the above described principles by controlling the opening and closing times of the valve 13. It should hence be understood that “a controllable oxygen valve for regulating a flow of oxygen through the oxygen inlet 17” as used herein may be any of a control valve, an on-off valve or any other type of valve that can be controlled to deliver oxygen to the gas delivery line 5 in accordance with the principles of the present disclosure.

With reference now to FIG. 2, the oxygen inlet 17 is preferably located downstream of the oxygen prefill compartment 9 along the gas delivery line 5. This way, the entire volume of the oxygen prefill compartment 9 can be used as a reservoir for oxygen that is to be delivered to the patient during a subsequent high-flow period of inspiration. Consequently, according to some embodiments, the oxygen prefill compartment 9 is arranged downstream of the air inlet 11 and upstream of the oxygen inlet 17. The oxygen inlet 17 is located on the same side of the flow generator 7 as the oxygen prefill compartment 9. Preferably but not necessarily, the oxygen prefill compartment 9 and the air inlet 17 are located upstream of the flow generator 7.

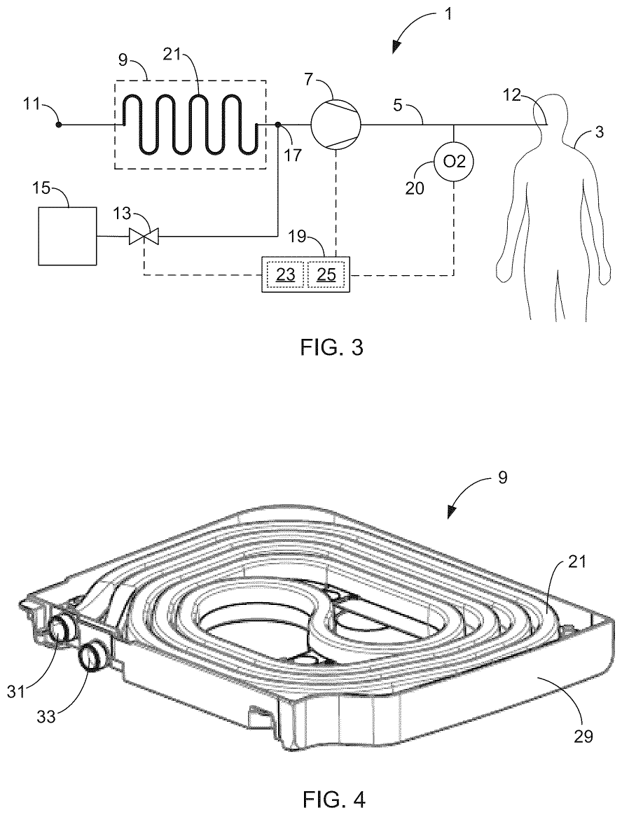

With reference now to FIG. 3, the oxygen prefill compartment 9 may comprise an elongated gas passageway 21 serving as an oxygen reflector and configured such that oxygen that flows into the oxygen prefill compartment 9 from the oxygen inlet 17 pushes gas contained in the gas passageway out from the oxygen prefill compartment 9 in the direction of the air inlet 11, and such that air that is drawn into the gas delivery line 5 by the under pressure generated by the flow generator 7 and flows into the oxygen prefill compartment 9 from the air inlet 11 pushes oxygen contained in the gas passageway out from the oxygen prefill compartment 9 in the direction of the patient 3. The configuration of the elongated gas passageway 21 with an upstream-end arranged in fluid communication with the air inlet 11 and a downstream-end arranged in fluid communication with the oxygen inlet 17 facilitates the formation of a well-defined gas front between the oxygen and the air in the oxygen prefill compartment 9. The well-defined gas front serves to minimise mixing of oxygen and air in the oxygen prefill compartment 9 while at the same time serving to virtually push the oxygen pillar and the air pillar back and forth in the oxygen prefill compartment 9.

The elongated gas passageway 21 of the oxygen prefill compartment 9 may have a length that is at least ten times a width or a diameter of the elongated gas passageway 21. This dimensional relation facilitates laminar flow through the oxygen prefill compartment, which in turn facilitates preservation of the well-defined gas front between the oxygen and air in the oxygen prefill compartment 9 and further reduces gas mixing within the compartment. The dimensions of the elongated gas passageway 21 may be adapted to the respiratory capacity of the patient 3 and the flow profile of the inspiratory flow generated by the flow generator 7. The length of the gas passageway 21 may, in some examples, be in the range of 0.5 to 4 meters. The width or diameter of the gas passageway may, in some examples, be in the range of 8 to 25 millimetres. The total volume of the gas passageway 21 may, in some examples, be in the range of 200-3000 ml, 200-2000 ml, 250-1500 ml, 300-1000 ml or 400-800 ml. In some embodiments, where the oxygen prefill compartment 9 is adapted to fit as many patient categories as possible, the total volume of the gas passageway 21 may be 800-1200 ml. By enabling connection of differently dimensioned oxygen prefill compartments in line with the gas delivery line 5, the oxygen prefill compartment 9 can be adapted to different patient requirements in an easy and effective way. In an exemplary scenario with ventilation of an adult patient, the gas passageway 21 may have a volume of approximately 1 litre and a length of approximately 2.5 metres.

In order for the ventilation system 1 to be able to deliver 100% oxygen to the patient 3 during inspiration, the oxygen prefill compartment 9 should have a minimum volume corresponding to the volume of breathing gas delivered during the high-flow period of inspiration and delivered at an inspiratory flowrate exceeding the maximum oxygen flowrate deliverable via the oxygen valve 13. This volume, hereinafter referred to as the top-flow volume, corresponds to the gas volume withdrawn from the oxygen prefill compartment during the high-flow period of an inspiration phase if the oxygen valve 13 is kept fully open to deliver a maximum flow of oxygen deliverable via the oxygen valve 13. Preferably, to account for minor mixing of air and oxygen at or near the gas front between the air and oxygen gas pillars, the volume of the oxygen prefill compartment 9 should be somewhat larger than the high-flow volume.

The gas passageway 21 may be a length of tubing, such as a length of tubing commonly used in ventilator circuits. The tubing may be folded or wounded to minimise the space occupied by the oxygen prefill compartment 9 in the ventilation system 1, and optionally be kept in a casing adapted to maintain the shape of the tubing. In other embodiments, the gas passageway 21 may be constituted by a hollow gas duct manufactured through, e.g., a moulding process or an additive manufacturing technique, which gas duct is fitted in a casing to form an oxygen prefill compartment component configured to be connected in line with the gas delivery line 5.

FIG. 4 illustrates an exemplary embodiment of such a component. In this embodiment, the oxygen prefill compartment 9 comprises an elongated gas passageway 21 in form of a hollow rigid gas duct. The gas duct is fitted in a casing 29 comprising an air inlet port 31 for receiving air from the air inlet 11 and an oxygen inlet port 33 for receiving oxygen from the oxygen inlet 17. The air inlet port 31 and the oxygen inlet port 33 are fluidly coupled to each other via the hollow rigid gas duct extending between the air inlet port 31 and the oxygen inlet port 33, within the casing 29. The oxygen prefill compartment is detachably connectable in line with the gas delivery line 5 between the air inlet 11 and the oxygen inlet 17.

The rigid gas duct constituting the gas passageway 21 of the oxygen prefill compartment 9 may be designed in various ways in order to provide a well-defined gas front between the air and oxygen pillars moving back and forth within the gas duct. The gas duct may have a substantially circular cross-section or it may be rectangular with rounded corners, or it may be oval or nearly oval. The cross section area of the duct, i.e. in the plane perpendicular to the flow direction, is preferably in the range of 300 to 450 mm2, more preferably in the range of 350 to 400 mm2, and most preferably about 370 mm2. The length and total volume of the gas duct may be the same as discussed above in relation to the gas passageway 21.

In the illustrated example, the rigid gas duct has an integrated one-piece moulded structure. The gas duct may be a single continuous gas duct comprising straight and curved portions. The rigid gas duct may have a shape resembling the shape of a folded tube. The air inlet port 31 and the oxygen inlet port 33 may be positioned adjacent to each other on a same side of the casing 29 and the free ends of the folded gas duct may be connected to a respective one of the air inlet port 31 and the oxygen inlet port 33. The two portions of the folded gas duct extending from the air inlet port 31 and the oxygen inlet port 33, respectively, may run alongside each other within the casing 29 and be spirally wound inwards towards a centre of the casing where the two portions come together, as best illustrated by the drawing.

The present disclosure also relates a method for controlling an oxygen content of oxygen-containing breathing gas delivered to a patient by the ventilation system comprising a flow generator for generating a flow of air from an air inlet of a gas delivery line towards the patient, via an oxygen prefill compartment of the gas delivery line, and a controllable oxygen valve for regulating a flow of oxygen through an oxygen inlet of the gas delivery line, which oxygen inlet is arranged in fluid communication with the oxygen prefill compartment. In accordance with the teachings of the present invention, the method comprises a step of prefilling the oxygen prefill compartment with a volume of oxygen that is to be delivered to the patient during a subsequent high-flow period of an inspiration phase.

As discussed above, the controller 19 of the ventilation system 1 is typically configured to control the oxygen valve 13 such that the oxygen prefill compartment is prefilled with oxygen only when other control strategies have failed to make a mean oxygen concentration delivered to the patient 3 substantially correspond to a set target oxygen concentration.

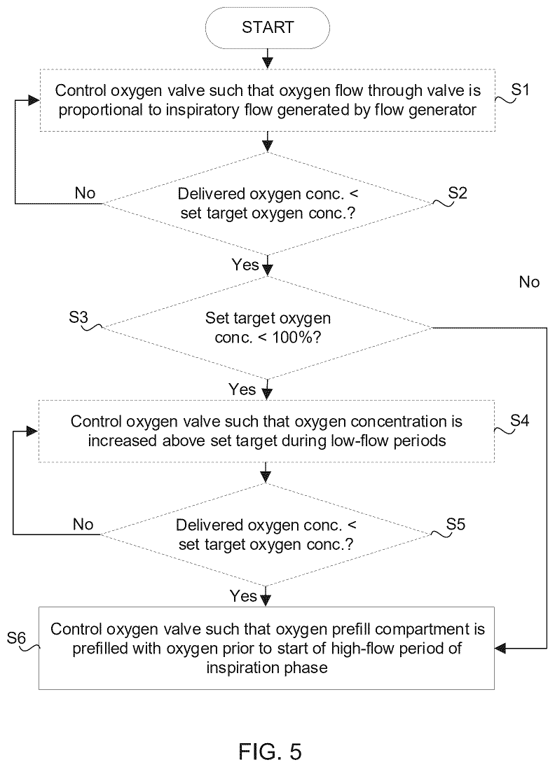

FIG. 5 is a flowchart illustrating a method comprising steps corresponding to the different oxygen valve control strategies discussed above with reference to FIG. 1. The method is hence a computer-implemented method performed by the controller 19 of the ventilation system 1 upon execution of a computer program by the processor 23. The method aims at controlling the oxygen valve 13 such that the mean oxygen concentration of breathing gas delivered to the patient 3 substantially corresponds to a set target oxygen concentration.

In a first step, S1, the controller 19 controls the oxygen valve 13 in accordance with a proportional control strategy such that the flow of oxygen through the oxygen valve 13 is proportional to a current inspiratory flow generated by the flow generator 7. This way, a constant oxygen concentration substantially corresponding to the set target oxygen concentration will be delivered to the patient 3 at any given time of the inspiration phase as long as the oxygen valve 13 is able to deliver the proportional flow required to reach the set target oxygen concentration.

If, in a step S2, the controller 19 detects that the mean oxygen concentration delivered to the patient during inspiration falls below the set target oxygen concentration, which is most likely due to an inability of the oxygen valve 13 to deliver a proportionate oxygen flow during the high-flow period of inspiration, the method proceeds to step S3.

In step S3, the controller 19 checks whether the set target oxygen concentration is less than 100%. If so, in accordance with the current proportional control strategy, the oxygen flow delivered through the oxygen valve 13 during low-flow periods of inspiration is less than the inspiratory flow generated by the flow generator 7. This means that it may still be possible to make the mean oxygen concentration delivered to the patient during the course of an inspiration phase meet the set target oxygen concentration by increasing the oxygen flow during the low-flow periods, i.e. by increasing the oxygen concentration during low-flow periods above the set target oxygen concentration, whereby the method proceeds to step S4. If, on the other hand, the set target oxygen concentration is 100%, the flow of oxygen delivered during low-flow periods already corresponds to the inspiratory flow rate generated by the flow generator 7 and the method proceeds to step S6.

In step S4, the controller 19 applies a control strategy according to which the breathing gas is boosted with oxygen during low-flow periods of ventilation. This is achieved by the controller 19 by controlling the oxygen valve 13 such that the flow of oxygen through the oxygen valve 13 is increased above the proportionate oxygen flow dictated by the previous proportional control strategy, thereby ensuring that an oxygen concentration above the set target oxygen concentration is delivered during low-flow periods of ventilation.

If, in a step S5, the controller 19 detects that the mean oxygen concentration delivered to the patient 3 during inspiration falls below the set target oxygen concentration, which is most likely due to the fact that the increase in oxygen concentration during low-flow periods is not enough to compensate for the inability of the oxygen valve 13 to deliver high oxygen concentrations during high-flow periods of inspiration, the method proceeds to step S6.

In step S6, the controller 19 controls the oxygen valve 13 such that the oxygen prefill compartment 9 is prefilled with oxygen prior to start of the high-flow period of one or more inspiration phases, in accordance with the teachings of the present disclosure.

The person skilled in the art realizes that the present disclosure is not limited to the embodiments described above. The person skilled in the art further realizes that modifications and variations are possible within the scope of the appended claims.

1. A ventilation system for supplying oxygen-containing breathing gas to a patient via a gas delivery line comprising a flow generator for generating a flow of air from an air inlet of the gas delivery line towards the patient, the ventilation system comprising:
an oxygen prefill compartment forming a part of the gas delivery line, arranged downstream of the air inlet such that air from the air inlet flows through the oxygen prefill compartment on its way towards the patient;
a controllable oxygen valve for regulating a flow of oxygen through an oxygen inlet of the gas delivery line, which oxygen inlet is arranged in fluid communication with the oxygen prefill compartment, and
a controller for controlling the oxygen valve such that a mean oxygen concentration of breathing gas delivered to the patient during inspiration substantially corresponds to a set target oxygen concentration,
wherein the oxygen prefill compartment and the oxygen inlet are arranged upstream of the flow generator along the gas delivery line, and wherein the controller is configured to control the oxygen valve such that the oxygen prefill compartment is prefilled with a volume of oxygen prior to start of a high-flow period of an inspiration phase, during which the gas flow generated by the flow generator exceeds a maximum flow of oxygen deliverable via the oxygen valve.

2. The ventilation system of claim 1, wherein the oxygen prefill compartment comprises an elongated gas passageway serving as an oxygen reflector configured
such that oxygen that flows into the oxygen prefill compartment from the oxygen inlet pushes air contained in the gas passageway out from the oxygen prefill compartment in the direction of the air inlet, and such that air that flows into the oxygen prefill compartment from the air inlet pushes oxygen contained in the gas passageway out from the oxygen prefill compartment in the direction of the patient.

3. The ventilation system of claim 2, wherein the oxygen inlet is arranged downstream of the elongated gas passageway, or in a downstream end of the elongated gas passageway.

4. The ventilation system of claim 2, wherein the elongated gas passageway of the oxygen prefill compartment has a length that is at least ten times a width or a diameter of the elongated gas passageway.

5. The ventilation system of claim 2, wherein the elongated gas passageway of the oxygen prefill compartment has a volume of 200-3000 ml.

6. (canceled)

7. The ventilation system of claim 1, wherein the controller is configured to control the oxygen valve such that the oxygen prefill compartment is prefilled during a low-flow period during which the gas flow generated by the flow generator is lower than the maximum flow of oxygen deliverable via the oxygen valve.

8. The ventilation system of claim 7, wherein the low-flow period occurs during any of, or any combination of, a final phase of an inspiration phase, an initial phase of an inspiration phase, and an expiration phase.

9. The ventilation system of claim 1, wherein the controller is configured to control the oxygen valve such that the oxygen prefill compartment is prefilled with oxygen prior to start of a high-flow period of at least one inspiration phase in response to a failure of making the mean oxygen concentration substantially correspond to the set target oxygen concentration by controlling the oxygen valve such that the oxygen concentration of the breathing gas is increased above the set target oxygen concentration during at least one low-flow period of at least one inspiration phase.

10. A method for controlling an oxygen content of oxygen-containing breathing gas delivered to a patient by a ventilation system comprising a flow generator for generating a flow of air from an air inlet of a gas delivery line towards the patient, via an oxygen prefill compartment of the gas delivery line, and a controllable oxygen valve for regulating a flow of oxygen through an oxygen inlet of the gas delivery line, which oxygen inlet is arranged in fluid communication with the oxygen prefill compartment, wherein the oxygen prefill compartment and the oxygen inlet are arranged upstream of the flow generator along the gas delivery line, the method comprising the steps of:
prefilling the oxygen prefill compartment with a volume of oxygen prior to start of a high-flow period of an inspiration phase, during which the gas flow generated by the flow generator exceeds a maximum flow of oxygen deliverable via the oxygen valve.

11. The method of claim 10, wherein the oxygen inlet is arranged downstream of an elongated gas passageway of the oxygen prefill compartment, or in a downstream end of the elongated gas passageway, and wherein the oxygen prefill compartment is prefilled during a low-flow period in which the gas flow generated by the flow generator is lower than the maximum flow of oxygen that can be delivered via the oxygen valve.

12. The method of claim 11, wherein the low-flow period occurs during any of, or any combination of, a final phase of an inspiration phase, an initial phase of an inspiration phase, and an expiration phase.

13. The method of claim 10, wherein the step of prefilling the oxygen prefill compartment prior to start of the high-flow period of the at least one inspiration phase is performed in response to a failure of making the mean oxygen concentration substantially correspond to the set target oxygen concentration by increasing the oxygen concentration of the breathing gas above the set target oxygen concentration during at least one low-flow period of at least one inspiration phase.

14. A computer program for controlling an oxygen content of oxygen-containing breathing gas delivered to a patient by a ventilation system comprising:
a flow generator for generating a flow of air from an air inlet of a gas delivery line towards the patient;
an oxygen prefill compartment forming part of the gas delivery line, arranged downstream the air inlet such that air from the air inlet flows through the oxygen prefill compartment on its way towards the patient;
a controllable oxygen valve for regulating a flow of oxygen through an oxygen inlet of the gas delivery line, which oxygen inlet is arranged in fluid communication with the oxygen prefill compartment, and
a controller for controlling the oxygen valve such that a mean oxygen concentration of breathing gas delivered to the patient during inspiration substantially corresponds to a set target oxygen concentration,
wherein the computer program comprises computer-readable instructions which, when executed by a processor of the controller, causes the controller to perform the method of claim 10.

15. A computer program product comprising a non-transitory data storage medium storing the computer program of claim 14.
